SEQUENCE LISTING (3 sequences)

SEQ ID NO: 1 - protein, 11 aa, Artificial Sequence
  Synthetically generated peptide
KEAPPAPPQSP

SEQ ID NO: 2 - protein, 12 aa, Artificial Sequence
  Synthetically generated peptide
GKEAPPAPPQSP

SEQ ID NO: 3 - protein, 17 aa, Artificial Sequence
  Synthetically generated peptide
RRRAAEELDSRAGSPQL

(19) United States
(12) Patent Application Publication
(10) Pub. No.: US 2006/0204980 A1
(43) Pub. Date: Sep. 14, 2006

(54) Colorectal cancer therapies

(76) Inventors: Dario C. Altieri, Worcester, MA (US); Jagadish Ghosh, Worcester, MA (US)

Correspondence Address: FISH & RICHARDSON PC, P.O. BOX 1022, MINNEAPOLIS, MN, 55440-1022, US

(21) Appl. No.: 11/313,901

(22) Filed: Dec. 21, 2005

Related U.S. Application Data
(60) Provisional application No. 60/639,742, filed on Dec. 28, 2004.

(51) Int. Cl. C12Q 1/68; A61K 38/10; A61K 31/4439; A61K 31/426; A61K 31/404; A61K 31/381; A61K 31/12; A61K 33/00

(52) U.S. Cl. 435/6; 424/722; 514/14; 514/15; 514/414; 514/340; 514/369; 514/438; 514/686

(57) ABSTRACT

Methods related to the targeted treatment of p53⁺ colorectal cancer cells, and methods of screening for new anti-tumor agents, are described.

### CLAIM OF PRIORITY

This application claims priority under 35 USC §119(e) to U.S. Provisional Patent Application Ser. No. 60/639,742, filed on Dec. 28, 2004, the entire contents of which are hereby incorporated by reference.

### FEDERALLY SPONSORED RESEARCH OR DEVELOPMENT

This invention was made with Government support under Grant Nos. CA78810, HL54131, and CA90917 awarded by the National Institutes of Health. The Government has certain rights in the invention.

### TECHNICAL FIELD

This invention relates to therapies for p53⁺ colorectal cancer.

### BACKGROUND

Targeted, or rational, cancer therapy is based on the recognition that all cancers are not created equal. Cancer development is a multi-step process involving a variety of cellular factors. These cellular factors, and the mechanisms by which they act, can vary according to (i) the type of tumor, (ii) the stage of tumor progression, (iii) and the individual suffering from a tumor. Biologically-based, rational cancer therapies recognize and target specific patient populations based on one or more of these criteria, i.e., tumor type, stage of tumor progression, and a patient's genetic background. By targeting specific populations, targeted cancer therapies are expected to increase the efficacy and/or decrease the side effects of treatment, relative to non-targeted therapeutics.

### SUMMARY

The new methods described herein are based, in part, on the discovery that inhibition of glycogen synthase kinase 3β (GSK3β) in colorectal cancer cells that express functional p53 protein (i.e., p53⁺ colorectal cancer) activates p53-dependent apoptosis and antagonizes colorectal tumor growth. The discovery has led to new rational anti-colorectal cancer therapies that use one or more GSK3β inhibitors to treat individuals with p53⁺ colorectal cancer, screening methods to identify subjects with colorectal cancer that are candidates for rational treatment with one or more GSK3β inhibitors, and screening assays for the identification of candidate compounds useful in the targeted therapy of p53⁺ colorectal cancer. By targeting individuals with p53⁺ colorectal cancer, the new ways of using GSK3β inhibitors to treat cancer are likely to be more therapeutically efficacious and more cost efficient, because the new methods avoid using GSK3β inhibitors to treat patients having p53⁻ colorectal cancer in which the drugs cannot induce p53-dependent apoptosis.

New methods are described herein for treating individuals with colorectal cancer. The new methods include selecting an individual whose colorectal cancer cells express functional p53 protein (e.g., selecting the individual on the basis that he or she has colorectal cancer, and the cancerous cells express functional p53 protein) and administering to the individual a pharmaceutical composition including an effective amount of a GSK3β inhibitor. An individual can be selected because the individual's medical history or the individual's colorectal tissue indicates (a) that the individual has colorectal cancer and (b) that the colorectal cancer tissue expresses functional p53 protein.

In one aspect, the invention provides methods for treating individuals with colorectal cancer. The methods include determining whether colorectal cancer cells of the individual express functional p53 protein; and, if the cells express functional p53 protein, administering to the individual a pharmaceutical composition comprising an effective amount of a glycogen synthase kinase 3β (GSK3β) inhibitor. In some embodiments, determining whether colorectal cancer cells of the individual express functional p53 protein includes or constitutes reviewing the individual's medical history. In some embodiments, determining whether colorectal cancer cells of the individual express functional p53 protein includes identifying an individual suffering from colorectal cancer; obtaining from the individual a sample comprising colorectal cancer cells; and assaying expression of functional p53 protein in the cells in the sample.

Determining whether colorectal cancer cells express functional p53 protein can include, for example, detecting the presence of mutant or wild-type forms of a p53 gene or protein, and/or assaying one or more functions of p53 protein in the cells, e.g., assaying p53 transcription factor activity in the cells.

Also described herein are new methods for screening individuals to determine whether they are candidates for treatment with GSK3β inhibitors. For example, if screening an individual's colorectal cancer cells indicates that these cells express functional p53 protein, then the individual should be classified as a candidate for treatment with GSK3β inhibitor. On the other hand, if the screen indicates that the individual's colorectal cancer cells do not express functional p53 protein, then the individual can be classified as someone who is not a candidate for colorectal cancer treatment with a GSK3β inhibitor.

In the new therapies for p53⁺ colorectal cancer described herein, an individual can be treated with one or more of the following GSK3β inhibitors: Purvalanol A, olomoucine; lithium chloride (LiCl), alsterpaullone, and kenpaullone. Other GSK3β-inhibitors that are useful in the treatments described herein include benzyl-2-methyl-1,2,4-thiadiazolidine-3,5-dione (TDZD-8); 2-thio(3-iodobenzyl)-5-(1-pyridyl)-[1,3,4]-oxadiazole (GSK3 inhibitor II); 2,4-dibenzyl-5-oxothiadiazolidine-3-thione (OTDZT); (2′Z,3′E)-6-Bromoindirubin-3′-oxime (BIO); α-4-Dibromoacetophenone (i.e., Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), H-KEAPPAPPQSpP-NH2 (L803; SEQ ID NO:1); Myr-N-GKEAPPAPPQSpP-NH2 (L803-mts; SEQ ID NO:2); and indirubins. Exemplary indirubins include indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin; and 5-bromoindirubin. Other GSK3β-inhibitors that can be used in the treatment methods described herein are known in the art.

In another aspect, the invention also features methods for identifying candidate therapeutics that are useful in the targeted treatment of p53⁺ colorectal cancer. In one screen for candidate therapeutics, a test compound is contacted to a sample including GSK3β. A sample can be a cell-free sample, or a sample can include colorectal cancer cells (or model cells of colorectal cancer) that express GSK3β. GSK3β activity is then assayed in the sample. Inhibition of GSK3β activity in the sample indicates that the test compound is a candidate therapeutic compound for treating p53⁺ colorectal cancer. Another method of identifying candidate therapeutics useful in the targeted treatment of p53⁺ colorectal cancer includes contacting a test compound to a sample including one or more p53⁺ colorectal cancer cells and assaying for activation of p53 apoptotic signaling in the cell. Exemplary p53 apoptotic signals that can be assayed include p53 expression; increased expression of the cyclin-dependent kinase inhibitor p21^{wild-type p53 activated-fragment 1 (Waf1)/cell cycle inhibitory protein (Cip1)}; increased p14^{ADP-Ribosylation Factor (ARF)} expression; and decreased expression of Mouse Double Minute 2 (MDM2), e.g., the human homolog of MDM2. Activation of p53 signaling in the sample indicates that the test compound is a candidate therapeutic compound for treating p53⁺ colorectal cancer. Starting test compounds include, but are not limited to, small molecules such as indirubins; paullones; and purvalanols.

p53 protein, as used herein, refers to human and non-human animal tumor suppressor proteins that (i) are encoded by a p53 gene and (ii) can induce apoptosis when functional. A p53 gene, as used herein, refers to genomic material that expresses a p53 protein, including the TP53 gene in humans and genes that encode a p53 protein in other animals. “Functional p53,” as used herein, refers to a p53 protein that is capable of participating in p53-mediated apoptosis. Assays for functional p53 protein are described herein and include detecting expression of wild-type p53 protein; assaying for p53 activity, e.g., transcription factor activity; and/or detecting the presence of a wild-type p53 gene and/or mRNA.

p53+, as used herein, refers to cells, or individuals with cells, that express functional p53 protein. On the other hand, p53−, as used herein, refers to cells, or individuals with cells, that do not express functional p53 protein.

As used herein, “individual(s)” refers to humans and non-human animals including, but not limited to, dogs, cats, horses, cows, pigs, goats, sheep, rats, mice, monkeys, fish, and birds.

As used herein, “individuals with colorectal cancer” include individuals suffering from colon cancer, rectal cancer, or cancer of both the colon and the rectum.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description, drawings, and from the claims.

### BRIEF DESCRIPTION OF FIGURES

FIG. 1 is a histogram showing the GSK3β inhibitory activity of Purvalanol A relative to established GSK3β inhibitors thiadiazolidinone-8 (TDZD) and LiCl.

FIG. 2A is a series of images of immunoblots for the indicated proteins, which were harvested from cells treated with vehicle or Purvalanol A.

FIG. 2B is a series of images showing immunoblots for the indicated proteins, which were harvested from cells treated with vehicle, Olomoucine, or Asterpaullone.

FIG. 2C is an autoradiograph image showing the results of an H1 histone kinase assay from cells treated with vehicle or Purvalanol A.

FIG. 2D is a graph quantifying the results of flow cytometry experiments on control cells and cells transfected with a dominant negative mutant of p34^{cdc2}. The percentage of cells with G2/M DNA content is indicated on each graph.

FIG. 2E is a set of two immunoblot images showing that a dominant negative mutant of p34^{cdc2} does not affect p53 expression.

FIG. 3A is a set of images of immunoblots for the indicated proteins, which were harvested from cells treated with a vehicle control or the indicated GSK3β inhibitors.

FIG. 3B is a series of images of immunoblots for the indicated proteins, which were harvested from cells treated with a vehicle control or the indicated drugs.

FIG. 4A is a series of images of immunoblots for the indicated proteins, which were harvested from cells expressing the indicated heterologous proteins.

FIG. 4B is a series of images of immunoblots for the indicated proteins, which were harvested from cells having wild-type or null alleles of GSK3β.

FIG. 5A is a series of images of immunoblots for the indicated proteins, which were harvested from cells transfected with a control vector or a vector encoding β-catenin.

FIG. 5B is a series of images of immunoblots for the indicated proteins, which were harvested from cells transfected with a control vector or a vector encoding a dominant negative mutant of T-cell factor-4 dominant negative (TCF-4 DN).

FIG. 5C is a series of images of immunoblots for the indicated proteins, which were harvested from cells treated with a vehicle control or the indicated drugs.

FIG. 5D is a series of images of immunoblots for the indicated proteins, which were harvested from cells treated with a vehicle control or the indicated drugs.

FIG. 6A is a series of graphs quantifying the results of flow cytometry experiments on cells that having wild-type or null alleles of p53, and which were treated with vehicle control or the indicated concentrations of Purvalanol A. The percentage of cells with hypodiploid content is indicated on each graph.

FIG. 6B is a set of images of immunoblots for the indicated proteins, which were harvested from cells treated with Purvalanol A for the indicated amount of time.

FIG. 6C is a series of multiparametric flow cytometry results for cells with the indicated p53 genotype, which were treated with a vehicle control or the indicated drug.

FIG. 6D is a series of graphs quantifying the results of flow cytometry experiments on cells that were (a) transfected with the indicated plasmid vectors and (b) treated with vehicle control or Purvalanol A. The percentage of cells with hypodiploid content is indicated on each graph.

FIG. 6E is a series of graphs (four left hand panels) and images of immunoblots (two right hand panels). The graphs quantify the results of flow cytometry experiments on cells that were (a) transfected with the indicated plasmid vectors and (b) treated with vehicle control or Purvalanol A. The percentage of cells with hypodiploid content is indicated on each graph. Protein samples in the immunoblots were harvested from cells transfected with the indicated plasmid vectors and were probed using an antibody that binds the indicated proteins.

FIG. 7 is a graph comparing the change in tumor volume over time of xenografted tumors in mice treated with Purvalanol A, vehicle control, or no treatment.

### DETAILED DESCRIPTION

The new methods disclosed herein are based, in part, on the discovery that structurally unrelated inhibitors of serine/threonine kinase p34^{cell division cycle 2 (cdc2)} potently elevate p53 levels in colorectal cancer cells, induce p53-dependent apoptosis, and antagonize tumor growth. As described in the Examples below, a sustained elevation of transcriptionally active p53 was observed in (p53^{+/+}) colon cancer cells treated with Purvalanol A, olomoucine, alsterpaullone, thiadiazolidinone-8 (TDZD), and LiCl. This elevation was not due to deregulation of mitosis, but rather to efficient inhibition of glycogen synthase kinase 3β (GSK3β). Pharmacological inhibitors of GSK3β or a GSK3β dominant negative mutant reproduced the increase in p53 expression and upregulation of p21^{Waf1/Cip1}. Levels of p53 were constitutively elevated in GSK3β^{-/-} cells, consistent with the discovery that GSK3β constitutively elevated. A TCF-4 dominant negative mutant that interrupts β-catenin signaling counteracted p53 and p21^{Waf1/Cip1} induction after GSK3β inhibition. Blockade of GSK3β was associated with elevation of the p14^{ARF} tumor suppressor, and attenuation of the negative p53 regulator, MDM2. Activation of the GSK3β-p53 axis resulted in p53-dependent apoptosis, which was contributed to by downregulation of survivin and inhibition of early tumor growth in vivo. These lines of evidence show that inhibition of GSK3β in colorectal cancer cells can activate p53-dependent apoptosis and antagonize tumor growth.

Targeted Therapy of Colorectal Cancer

Described herein are new methods for the targeted therapy of colorectal cancer. Generally, the methods include (i) selecting an individual with colorectal cancer cells that express functional p53 protein and (ii) administering to the individual a pharmaceutical composition including a GSK3β inhibitor.

In some embodiments, the methods include (i) selecting an individual suffering from colorectal cancer and (ii) determining if the individual's colorectal cancer cells express functional p53 protein. If these cells express functional p53 protein, then the individual is a candidate for treatment with a GSK3β inhibitor, and the method further includes (iii) administering to the individual a pharmaceutical composition including a GSK3β inhibitor. If the individual colorectal cancer cells do not express functional p53 protein, then the individual is not a candidate for treatment with a GSK3β inhibitor, and the method optionally includes (iii) pursuing a course of treatment that does not include administering a pharmaceutical composition including a GSK3β inhibitor.

Candidates for treatment with a GSK3β inhibitor are individuals with colorectal cancer cells that express functional p53 protein. On the other hand, the absence of functional p53 protein in an individual's colorectal tumor generally indicates that the individual is not a candidate for treatment with a GSK3β inhibitor.

Additional methods of identifying individuals who are candidates for treatment with a GSK3β inhibitor are disclosed herein. In these new methods, a colorectal cancer cell from an individual is (i) exposed to a GSK3β inhibitor and (ii) assayed for the presence of one or more of the following activities: increased p53 expression, decreased survivin expression, and increased p21 expression. Methods for performing such assays are known in the art and include quantitative PCR, gel electrophoresis, western blotting, and quantitative protein and DNA chips. Exemplary methods are also described in Examples 2-4.

If the colorectal cancer cell exhibits one or more of these activities, then the individual is classified as a candidate for treatment with a GSK3β inhibitor. In other new methods, colorectal cancer cells from the same individual are placed in culture media. Some of the cancer cells are contacted with a GSK3β inhibitor, and cultured under conditions that allow the cells to proliferate. If the GSK3β inhibitor inhibits proliferation and/or induces apoptosis of the contacted cancer cells, e.g., relative to cells that are not contacted with an inhibitor, then the individual is a candidate for treatment with GSK3β inhibitor.

Identifying Individuals Suffering from Colorectal Cancer

Screens for identifying individuals with colorectal cancer are known in the art. For example, screens for colorectal cancer include: fecal occult blood test (FOBT), which checks for blood in the stool, digital rectal exam (DRE), which checks for tactile abnormalities in the rectum, sigmoidoscopy, which looks for visual abnormality in the rectum and lower part of the colon, colonoscopy, which allows visualization of the rectum and entire colon, and double contrast barium enema (DCBE), which allows radiographic examination of the rectum and colon. Frequently, a biopsy or polypectomy of abnormal colorectal tissue is examined to confirm that the tissue is cancerous.

Individuals with colorectal cancer can be classified according to cancer stage scales, such as the Dukes, Astler-Coller, and AJCC/TNM scales. An individual's grade of cancer indicates the degree of de-differentiation the cancer cells have undergone, i.e., how much the tumor's cells still retain the characteristics of a colon or rectal cell. Stage groupings are indicative of person's overall disease stage. In some systems, stage groupings are expressed as Roman numerals from 0 (the earliest stage) to IV (the most advanced stage). In stage 0, the cancer is found only in the inner lining of the colon or rectum. In stage I, the cancer has spread to more of the inner wall of the colon or rectum. In stage II, the cancer has spread outside the colon or rectum to nearby tissue, but has not spread to the lymph nodes. In stage III, the cancer has spread to nearby lymph nodes but not to other parts of the body. In stage IV, the cancer has spread to other parts of the body. Colorectal cancer tends to spread to the liver and/or lungs. (Stages 0 and IV, just described, correspond to stages A and D, respectively, in the Duke scale). Further information on the screening, diagnosis, and staging of colorectal cancer can be found in Frei et al., Cancer Medicine, B C Decker Inc., Hamilton, Ontario (2003), incorporated by reference herein in its entirety.

Exemplary candidates for treatment with GSK3β inhibitors include individuals in stages I, II, and/or III of colorectal cancer. This is because functional p53 is often lost later stages of colon cancer, e.g., by stage IV. However, as explained below, individuals at any stage of the disease may be candidates for treatment with one or more GSK3β inhibitors, as long as their colorectal tumors express functional p53 protein.

Glycogen Synthase Kinase 3β (GSK3β)

GSK3β is a pleiotropic serine/threonine kinase originally recognized as a component of insulin signaling via inhibitory phosphorylation by Protein kinase B (PKB/Akt) (Pap et al., J. Biol. Chem., 273:19929-19932 (1998)). GSK3β was more recently implicated in multiple signal transduction networks influencing cell adhesion, cell cycle progression, body axis specification, and gene expression regulators (Cohen and Frame, Nat. Rev. Mol. Cell. Biol., 2:769-776 (2001) and Kim and Kimmel, Curr. Opin. Genet. Dev., 10:508-514 (2000). A role of GSK3β in cell survival has also been proposed, and inhibition of kinase activity has been linked to enhanced neuronal cell viability (Cross et al., J. Neurochem., 77:94-102 (2001)), or defective activation of Nuclear Factor Kappa B (NFκB)-dependent cell survival in the developing liver (Hoeflich et al., Nature, 406: 86-90 (2000)).

Tumor Suppressor p53

The tumor suppressor p53 is mutated in many human cancers. (see Hollstein et al., Science, 253:49-53, (1991)). The sequence of wild-type human p53 protein has been published, for example, at Accession No. NP_{—}000537.2 in the Entrez protein database, available through the National Center for Biotechnology Information (NCBI) web site. The coding sequence of p53 mRNA has also been published, for example, at Accession No. NM_{—}000546.2 in the Entrez nucleotide sequence database, available through the NCBI web site. Human p53 is expressed from the gene known as TP53, which maps to position 17p13.1 of chromosome 17. The complete genomic p53 coding sequence of human TP53 can be found at GenBank Accession No. AH002918.1, available through the NCBI web site. Corresponding p53 proteins have been identified in a number of non-human animals such as dog, cat, and mouse (Accession Nos., NP_{—}001003210.1; P41685.0, NP_{—}112251.1, AAK53397.1, respectively) as well as numerous other animals that can be found searching the Entrez database through the NCBI web site.

Assays for Functional p53 Protein

The methods described herein can include determining whether a cell expresses functional p53 protein.

Generally, cells that express functional p53 protein are cells that harbor wild-type p53 genes. Thus, determining whether a cell expresses functional p53 protein can include determining whether the cell harbors a functional p53 gene. A number of assays are known in the art for determining whether a cell harbors a functional p53 gene. In some methods described herein, one or more colorectal cancer cells from an individual are screened for expression of functional or non-functional forms of the p53 gene. Functional forms of the p53 gene are those that express functional gene product (p53 protein) suitable for inducing p53-dependent apoptosis. For reviews of p53-dependent apoptosis see, e.g., Haupt et al., J. Cell Sci., 116:4077-4085, 2003 and Friedman and Lowe, Oncogene, 22:9030-9040, (2003). Non-functional forms of the p53 gene do not express functional p53 gene product (i.e., because they express mutant gene product or no gene product at all). Screens for a functional or non-functional genomic p53 gene can include amplifying, in part or in its entirety, one or more genomic copies of the p53 gene using polymerase chain reaction (PCR). Screens for a functional or non-functional genomic p53 gene can include amplifying the coding sequence, in whole or in part, using reverse transcriptase-polymerase chain reaction (RT-PCR). Wild-type and/or mutant genomic copies of a p53 gene or coding sequence can be detected using a number of techniques, including, but not limited to, direct DNA sequencing (DS), denaturing high performance liquid chromatography (DHPLC), single-strand conformation polymorphism (SSCP), denaturing gradient gel electrophoresis (DGGE), heteroduplex analysis (HA), and fluorescent assisted mismatch analysis (FAMA). Mutant forms of the p53 gene can also be distinguished from wild-type forms of the gene by fluorescent in-situ hybridization (FISH) or Northern blot analysis.

The presence of functional p53 protein can also be assayed directly. For example, one or more colorectal cancer cells from an individual can be screened for functional or non-functional forms of the p53 protein. Mutant forms of p53 protein can be distinguished from wild-type forms of p53 using a number of techniques, including, but not limited to, Western blotting and ELISA. For example, non-functional forms of p53 can include truncations or other mutants identified because they migrate differently in a protein gel. In other examples, non-functional p53 protein mutants are identified by their lower binding affinity to anti-p53 antibodies raised against wild-type p53 peptides. Conversely, antibodies raised to certain mutant forms of p53 will not bind wild-type p53 proteins.

In some methods, the presence of functional p53 protein can be detected using as assay of p53 function as a transcription factor. For example, functional p53 protein can be detected by its ability to bind a p53 consensus sequences in a target DNA. As an exemplary readout, an electrophoretic mobility shift assay (EMSA) can show whether p53 binds to a p53 consensus sequence in target DNA by observing a shift in migration.

Commercial examples of p53 activity assays include DuoSet® IC p53 transcription factor activity assay from R&D Systems, Inc. (Minneapolis, Minn.). The DuoSet® IC ELISA kit can be used to measure active human and mouse p53. Briefly, a biotinylated double stranded (ds) oligonucleotide containing a consensus p53 binding site is incubated with nuclear extracts, e.g., nuclear extracts from the colorectal cancer cells from a subject. p53-ds oligonucleotide complexes are subsequently captured by an immobilized antibody specific for p53. After washing away unbound material, detection utilizing Streptavidin-HRP is performed. An unlabeled ds competitor oligonucleotide is provided with the kit to demonstrate the specificity of the assay.

Cells that express functional p53 protein are cells that, in response to p53-mediated apoptotic signals (i.e., signals that, in a cell expressing wild-type p53, would induce p53-mediated apoptosis; such signals include, but are not limited to, DNA damage), activate the p53-dependent apoptotic signaling cascade. The p53-dependent apoptotic signaling cascade is known in the art (see e.g., Vogelstein et al., Nature, 408:307-310 (2000); Haupt et al., J. Cell Sci., 116:4077-4085 (2003); and Friedman and Lowe, Oncogene, 22:9030-9040, (2003)). As used herein, cells that do not express functional p53 protein are cells that, in response to p53-mediated apoptotic signals, are not induced to undergo p53-dependent apoptosis. Cells that express functional p53 protein can thus be identified using an assay of p53-mediated apoptotic signalling, as known in the art and described herein.

Exemplary GSK3β Inhibitors

As described in the Examples below, Purvalanol A suppressed GSK3β activity in colorectal cancer cells more potently than known GSK3β inhibitors, LiCl and TDZD. Purvalanol A is a 2,6,9 trisubstituted competitive ATP inhibitor that has been previously characterized by its ability to block p34^{cdc2} activity at nanomolar concentrations (i.e., with nM IC₅₀). Purvalanol A has been shown to be equally effective at suppressing Cdk2 and p42/p44 MAP kinase in vivo (Knockaert et al., Oncogene, 21:6413-6424 (2002)). At variance with other p34^{cdc2} inhibitors, analysis of a battery of kinase substrates suggested that Purvalanol A was not active against GSK3β, in vitro (Bain et al., Biochem. J., 37:199-204 (2003)). Although it remains possible that Purvalanol A exerts its effect on GSK3β indirectly, the inhibition observed here may reflect optimal substrate accessibility in vivo, which may not be adequately reproduced by screenings with isolated recombinant proteins in vitro.

Other GSK3β inhibitors include olomoucine, lithium chloride (LiCl), and paullones such as alsterpaullone and kenpaullone. Still other GSK3β inhibitors include indirubins such as indirubin-5-sulfonamide, indirubin-5-sulfonic acid (2-hydroxyethyl)-amide indirubin-3-monoxime, indirubin-39-monoxime, 5-iodo-indirubin-39-monoxime, and 5,59-dibromoindirubin, as well as other indirubins with GSK3β inhibitory activity described in Leclerc et al., J. Biol. Chem., 276:251-260, 2001.

The following GSK3β inhibitors are available from Calbiochem, San Diego Calif.): (i) 4-Benzyl-2-methyl-1,2,4-thiadiazolidine-3,5-dione (TDZD-8); 2-thio(3-iodobenzyl)-5-(1-pyridyl)-[1,3,4]-oxadiazole (GSK3 inhibitor II); (ii) 2,4-Dibenzyl-5-oxothiadiazolidine-3-thione (OTDZT); (iii) (2′Z,3′E)-6-Bromoindirubin-3′-oxime (BIO); (iv) α-4-Dibromoacetophenone (i.e., Tau Protein Kinase I (TPK I) Inhibitor); (v) 2-Chloro-1-(4,5-dibromo-thiophen-2-yl)-ethanone, (vi) N-(4-Methoxybenzyl)-N′-(5-nitro-1,3-thiazol-2-yl)urea (AR-A014418), and (vii) H-KEAPPAPPQSpP-NH2 (L803; SEQ ID NO:1) or its cell-permeable derivative Myr-N-GKEAPPAPPQSpP-NH2 (L803-mts; SEQ ID NO:2). Other GSK3β inhibitors are disclosed in U.S. Pat. Nos. 6,417,185; 6,489,344; 6,608,063 and Published U.S. Applications Nos. 690497, filed Oct. 20, 2003; 468605, filed Aug. 19, 2003; 646625, filed Aug. 21, 2003; 360535, filed Feb. 6, 2003; 447031, filed May 28, 2003; and 309535 filed Dec. 3, 2002.

Screens for Identifying Candidate Compounds that Inhibit p53⁺ Colorectal Cancer

Described herein are methods for identifying candidate compounds, e.g., small organic or inorganic molecules (M.W. less than 1,000 Da), oligopeptides, oligonucleotides, carbohydrates, and antibodies that are useful in the rational treatment of p53⁺ colorectal cancer. In some methods a candidate compound is screened for its ability to inhibit GSK3β in colorectal cancer cells. Screens for GSK3β inhibitory activity include immunoprecipitation and GSK3β kinase assays, affinity chromatography, enzyme-linked immunosorbent assays (ELISA), p53 upregulation assays, p21^{Waf1/Cip1} upregulation assays, p14^{ARF} upregulation assay, and MDM2 downregulation assays.

Libraries of Test Compounds

In certain embodiments, screens for candidate compounds that target p53⁺ colorectal cancer use libraries of test compounds. As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds, e.g., heteroorganic or organometallic compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. In some cases libraries include classes of compounds with enhanced potential for having anti-colorectal cancer activity. Classes of compounds with enhanced potential include known GSK3β inhibitors and structurally similar compounds. For example libraries can include purvalanols, paullones, and indirubins, and chemically modified species of purvalanols, paullones, and indirubins, and compounds having similar structures thereto. A library can be designed and synthesized to cover such a class of chemicals.

The synthesis of combinatorial libraries has been reviewed (see, e.g., Gordon et al., J. Med. Chem., 37:1385 (1994); DeWitt and Czarnik, Acc. Chem. Res. 29:114 (1996); Armstrong et al., Acc. Chem. Res., 29:123-131 (1996); Ellman, J. A., Acc. Chem. Res., 29:132 (1996); Gordon et al., Acc. Chem. Res., 29:144 (1996); Lowe, G. Chem. Soc. Rev., 309 (1995), Blondelle et al., Trends Anal. Chem., 14:83 (1995); Chen et al., J. Am. Chem. Soc., 116:2661 (1994); U.S. Pat. Nos. 5,359,115, 5,362,899, and 5,288,514; and PCT Publication Nos. WO92/10092, WO93/09668, WO91/07087, WO93/20242, and WO94/08051).

Libraries of compounds can be prepared according to a variety of methods, some of which are known in the art. For example, a split-pool strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky, Principles of Peptide Synthesis, 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, pooled (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a biased library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The split-pool strategy can result in a library of peptides, e.g., modulators, which can be used to prepare a library of test compounds for use in the screens described herein. In another illustrative synthesis, a diversomer library is created by the method of DeWitt et al., Proc. Natl. Acad. Sci. U.S.A., 90:6909 (1993). Other synthesis methods, including the “tea-bag” technique, described in Houghten et al., Nature, 354:84 (1991), can also be used to synthesize libraries of compounds according to the subject invention.

Libraries of compounds can be screened to determine whether any members of the library have GSK3β inhibitory activity, and, if so, to identify the inhibitor. Methods of screening combinatorial libraries have been described. See, e.g., Gordon et al., J. Med. Chem., supra. Soluble compound libraries can be screened to isolate inhibitors of GSK3β, followed by identification of the isolated ligands by conventional techniques (e.g., mass spectrometry, NMR, and the like). Screens are described herein.

Medicinal Chemistry

Once a compound (or agent) of interest has been identified, standard principles of medicinal chemistry can be used to produce derivatives of the compound. Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a candidate compound or agent and measure the effects of the modification on the efficacy of the compound or agent to thereby produce derivatives with increased potency. For an example, see Nagarajan et al., J. Antibiot. 41:1430-8 (1988). Furthermore, if the biochemical target of the compound (or agent) is known or determined, the structure of the target and the compound can inform the design and optimization of derivatives. Molecular modeling software is commercially available (e.g., Molecular Simulations, Inc.) for this purpose.

Screens

Provided herein are methods for identifying compounds capable of inhibiting p53⁺ colorectal cancer. Although applicants do not intend to be bound by any particular theory as to the biological mechanism involved, such compounds are thought to (1) inhibit function of GSK3β (e.g., the ability to bind GSK3β, reduce GSK3β kinase activity, and/or GSK3β-mediated expression of apoptosis regulatory genes) and/or (2) reduce expression of the GSK3β gene.

In certain embodiments, screening for compounds capable of inhibiting p53⁺ colorectal cancer can include identifying from a group of test compounds those that (i) inhibit and/or bind to GSK3β, (ii) modulate GSK3β-mediated expression of apoptosis regulatory genes, and/or (iii) modulate transcription and/or translation of GSK3β. Test compounds that exhibit one or more of activities (i), (ii) or (iii) are referred to herein as “candidate compounds.” Screening assays can optionally include further testing candidate compounds for their ability to modulate proliferation of colorectal cancer cells in vitro or in vivo. Screening assays of the present invention may be carried out in whole cell preparations and/or in ex vivo cell-free systems.

In some methods, test compounds that are candidate compounds for inhibiting p53⁺ colorectal cancer can be identified by contacting a test compound to a sample that includes one or more p53⁺ colorectal cancer cells, and then screening for diminished GSK3β kinase activity in the cells. In one embodiment, a p53⁺ colorectal cancer cell is contacted with one or more test compounds, e.g., incubated with a test compound for 0.5, 1, 2, 3-5, or 6-84 hours, and GSK3β activity in the cell is evaluated. GSK3β kinase activity in a cell can be evaluated using a phosphorylation assay. In some phosphorylation assays, GSK3β is immunoprecipitated from a colorectal cancer cell, immunoprecipitated GSK3β is contacted with GSK3β substrate and a phosphate source, and the phosphorylation of substrate is detected. Substrates of GSK3β include glycogen synthase and peptide fragments thereof, e.g., Arg-Arg-Arg-Ala-Ala-Glu-Glu-Leu-Asp-Ser-Arg-Ala-Gly-pSer-Pro-Gln-Leu (SEQ ID NO:3) and other peptides commercially available from Upstate Biotechnology (Lake Placid, N.Y.).

When a labeled phosphate source is used, e.g., ³²P-labeled ATP, substrate phosphorylation can be evaluated by detecting the incorporation of labeled phosphate into substrate. For example, the amount of labeled phosphate that co-precipitates with a substrate can be detected. Alternatively, the amount of labeled phosphate that co-migrates with substrate through a protein gel can be detected. In other methods, phosphorylated substrate can be detected by a mobility shift assay that detects the differential migration of phosphorylated substrate relative to non-phosphorylated substrate in a protein gel. Phosphorylated substrate can also be detected by its differential affinity to anion-exchange resins, relative to un-phosphorylated substrate by, for example, using anion exchange chromatography or anion exchange beads. In another embodiment, diminished GSK3β kinase activity in a p53⁺ colorectal cancer cell can be detected by evaluating the phosphorylation of GSK3β substrates in the cell. For example, p53⁺ colorectal cancer cells can be metabolically labeled with labeled phosphate, and then phosphorylation of one or more GSK3β substrates (e.g., glycogen synthase) can be detected, e.g., by immunoprecipitating the one or more GSK3β substrates from the cell, and quantifying the labeled incorporated onto the substrate.

In another embodiment, test compounds that are candidate compounds for inhibiting p53⁺ colorectal cancer can be identified by contacting a test compound to a sample that includes one or more p53⁺ colorectal cancer cells, and then screening for compounds that modulate GSK3β-mediated expression of an apoptosis regulatory gene such as p53, p21^{Waf1/Cip1}, p14^{ARF}, or MDM2 in the cells. Methods for evaluating expression of an apoptosis regulatory gene include microarray expression analysis, Northern blot, quantitative reverse transcriptase polymerase chain reaction (RT-PCR), Western Blot, enzyme-linked immunosorbent assay ELISA, and immunoprecipitation. In one embodiment, test compounds that increase p53 expression are classified as candidate compounds. In another embodiment, test compounds that increase p21^{Waf1/Cip1} expression are classified as candidate compounds. In another embodiment, test compounds that increase p14^{ARF} expression are classified as candidate compounds. In another embodiment, test compounds that decrease MDM2 expression are classified as candidate compounds.

Binding of a test compound to cell-free sample that includes GSK3β can be detected, for example, in vitro by reversibly or irreversibly immobilizing GSK3β on a substrate, e.g., the surface of a well of a plate (e.g., 96-well polystyrene microtitre plate). For example, microtitre plates can be coated with GSK3β, or a fragment thereof, washed and blocked (e.g., with BSA) to prevent non-specific binding of test compounds to the plates. GSK3β is then cross-linked to the plate. Test compounds are added to the coated plate under a number of conditions (e.g., at 37° C. for 0.5-12 hours). The plate can then be rinsed and binding of the test compound to GSK3β can be detected by any of a variety of art-known methods. For example, an antibody that specifically binds to a GSK3β can be used in an immunoassay. If desired, the antibody can be labeled (e.g., fluorescently or with a radioisotope) and detected directly (see, e.g., West and McMahon, J. Cell Biol. 74:264 (1977)). Alternatively, a second antibody can be used for detection (e.g., a labeled antibody that binds to the anti-GSK3β antibody). Test compounds that bind to GSK3β can be detected by their ability to inhibit binding of antibody to immobilized GSK3β. In an alternative detection method, the test compound is labeled (e.g., with a radioisotope, fluorophore, chromophore, or the like), and the binding of a test compound to GSK3β is detected by detecting label that is immobilized on the substrate.

In still another embodiment, test compounds are immobilized on a substrate, e.g., to a microtitre plate as described above, incubated with a cell free sample that includes GSK3β (or a fragment thereof), washed, and the ability of GSK3β to bind to an immobilized test compound is detected. For example, GSK3β (or a fragment thereof) can be produced as a fusion protein with a protein that can be detected optically, e.g., green fluorescent protein or a variant thereof (which can be detected under UV light), and the ability of the fusion protein to bind the test compound is detected. Alternatively, GSK3β can be produced as a fusion protein with an enzyme having a detectable enzymatic activity, such as horseradish peroxidase, alkaline phosphatase, β-galactosidase, or glucose oxidase. Genes encoding all of these enzymes have been cloned and are available for use by skilled practitioners. If desired, the fusion protein can include an antigen, which can be detected and measured with a polyclonal or monoclonal antibody using conventional methods. Suitable antigens include enzymes (e.g., horse radish peroxidase, alkaline phosphatase, and β-galactosidase) and non-enzymatic polypeptides (e.g., serum proteins, such as BSA and globulins, and milk proteins, such as caseins). In these methods, the ability of GSK3β fusion protein to bind to a test compound is detected.

To identify polypeptides that bind to a GSK3β, a two-hybrid assays of protein/protein interactions can be used (see, e.g., Chien et al., Proc. Natl. Acad. Sci. USA, 88:9578 (1991); Fields et al., U.S. Pat. No. 5,283,173; Fields and Song, Nature, 340:245 (1989); Le Douarin et al., Nucleic Acids Research, 23:876 (1995); Vidal et al., Proc. Natl. Acad. Sci. USA, 93:10315-10320 (1996); and White, Proc. Natl. Acad. Sci. USA, 93:10001-10003 (1996)). Kits for practicing various two-hybrid methods are commercially available (e.g., from Clontech; Palo Alto, Calif.).

In certain other embodiments, the interaction of a GSK3β, or fragment thereof, and test compound is detected by fluorescence resonance energy transfer (FRET) between a donor fluorophore covalently linked to either GSK3β or the test compound and an acceptor fluorophore covalently linked to either GSK3β or the test compound, wherein the acceptor and donor fluorophore are not both linked to GSK3β or the test compound, and there is suitable overlap of the donor emission spectrum and the acceptor excitation spectrum to give efficient nonradiative energy transfer when the fluorophores are brought into close proximity through the GSK3β-test compound interaction.

In another embodiment of a method of identifying test compounds that decrease expression of a GSK3β, p53⁺ colorectal cancer cells are contacted with one or more test compounds and the cells are evaluated for decreased expression of GSK3β. In a related method, one or more test compound is contacted to a p53⁺ colorectal cancer cell that expresses recombinant GSK3β encoding, and the cells are evaluated for decreased expression of GSK3β. Expression of GSK3β can be measured, for example, by Northern blot, RT-PCR analysis, RNAse protection analyses, Western blot, enzyme-linked immunosorbent assay (ELISA), radioimmunoassay (RIA) and fluorescent activated cell sorting (FACS). The level of expression in the presence of the test molecule, compared with the level of expression in its absence, will indicate whether or not the test compound inhibits the expression of GSK3β.

Having identified a test compound as a candidate compound, the candidate compound can be further tested, e.g., in proliferation assays of p53⁺ colorectal cancer using in vitro or in vivo model systems. In vitro proliferation assays include contacting a candidate compound to a culture of p53⁺ colorectal cancer cells, e.g., HCT116 cells, and evaluating the ability of the candidate compound to induce apoptosis in and/or prevent proliferation of the cultured cells. In vivo tumor assays include administering a candidate compound to an animal model, e.g., a rodent, with a colorectal tumor or a predisposition to develop colorectal tumor, and subsequently evaluating the candidate compound's ability to inhibit tumor development or tumor proliferation in the animal. Animal models of colorectal cancer include animals with xenografted p53⁺ colorectal cancer cells. Other animal models include rodents with a genetic predisposition to develop p53⁺ colorectal tumors, e.g., mice bearing mutant forms of (i) adenomatous polyposis coli (APC) gene (e.g., a multiple intestinal neoplasia (APC^{Min}) mouse (see, e.g., Haigis et al., Proc. Nat'l. Acad. Sci. USA, 101:9769-9773 (2004)), (ii) mut-s homologue-2 (Msh2) gene (see, e.g., Kohonen-Corish et al., Cancer Research 62:2092-2097 (2002)), and/or (iii) MutL homologue-1 (Mlh1) gene (see, e.g., Cohen et al., Cell 85:1125-1134 (1996)). The C57BL/6J-Apc^{Min} mouse is available from Jackson Harbor Labs (Bar Harbor, Me.). Alternatively, an animal model can be exposed to carcinogenic chemicals such as dimethylhydrazine derivatives or heterocyclic amines, such as 2-amino-1-methyl-6-phenylimidazo[4,5-b]pyridine (PhIP), that have been reported to induce p53⁺ colorectal tumors in animal models.

Potential Mechanism of GSK3β-Mediated Anti-Colorectal Cancer Activity

Pharmacologic, molecular, and genetic evidence presented in the Examples below demonstrates that acute interference with GSK3β kinase activity results in rapid upregulation of transcriptionally active p53. The ability of p34^{cdc2} antagonists, particularly roscovitine and olomoucine, to induce expression of nuclear p53 has been described previously (Lu, et al., Oncogene, 20:3206-3216 (2001)). At variance with previous models, elevation of p53 by Purvalanol A, olomoucine, or alsterpaullone (i) did not follow the kinetics of p34^{cdc2} inhibition in vivo, (ii) was unaffected by a p34^{cdc2} dominant negative (DN) mutant, and (iii) was recapitulated by blockade of GSK3β with structurally different pharmacologic inhibitors or a kinase-dead DN mutant of GSK3β.

As disclosed herein, over-expression of β-catenin reproduced the induction of the p53 signaling cascade in colorectal cancer cells. A T-cell factor-4 dominant negative (TCF-4 DN) mutant counteracted this induction of transcriptionally active p53. Whereas accumulation of p53 via transfection or exposure to DNA damage resulted in downregulation of β-catenin (as reported in Sadot et al., Mol. Cell Biol., 21:6768-6781 (2001)), forced expression of β-catenin in colorectal cancer cells may be associated with stabilization of p53 through induction of the p14^{ARF} tumor suppressor gene. It has been previously shown that p14^{ARF} is dynamically induced by oncogenic signaling, and may potentially constitute one of the t-cell factor (TCF)/β-catenin downstream target genes. Although how p14^{ARF} influences p53 stability has not been completely elucidated, one potential pathway involves attenuation of p53 degradation either through partial destabilization of the negative p53 regulator, murine double minute 2 (MDM2) and/or nuclear retention of the p53-MDM2 complex. Consistent with these observations, Purvalanol A or a thiadiazolidinone (TDZD) resulted in p14^{ARF} induction and partial attenuation of MDM2 levels, which may synergistically cooperate in stabilizing p53 levels.

A role of GSK3β in cell survival has been investigated. Interestingly, in neuronal cells, GSK3β inhibition elicits a survival signal potentially through activation of the PI-3 kinase/Akt pathway (Pap et al., J. Biol. Chem., 273:19929-19932 (1998)). Furthermore, some studies have suggested that GSK3β inhibitors may be cytoprotective in neurodegenerative disorders (Carmichael et al., J. Biol. Chem., 277:33791-33798 (2002)).

The mechanisms underlying p53-induced apoptosis have been extensively investigated and are thought to involve both transcriptional and post-transcriptional mechanisms. A potential role of survivin in this pathway has emerged with the observations that p53 actively represses survivin gene expression either directly or through promoter squelching. Consistent with these findings, Purvalanol A induction of p53 resulted in rapid disappearance of survivin, whereas forced expression of survivin counteracted p53-induced apoptosis in Purvalanol A-treated cells, and survivin ablation by RNAi restored the sensitivity of p53^{-/-} cells to Purvalanol A-induced cell death. This is consistent with mechanistic data demonstrating that survivin and p53 operate in the same apoptotic pathway of mitochondrial-dependent cell death.

The biological relevance of these observations is highlighted by the anti-tumor activity of Purvalanol A, in vivo, which resulted in early inhibition of colorectal xenograft tumors and suppression of cell proliferation. The GSK3β-p53 axis described herein provides a target that can be rationally exploited for tailored treatment of colorectal cancer patients retaining wild-type p53 function.

The invention is further described in the following examples, which do not limit the scope of the invention described in the claims.

### EXAMPLES

In the examples that follow, a number of drugs, including Purvalanol A, were shown to acutely inhibit GSK3β in colorectal cancer cells. GSK3β inhibition in colorectal cancer cells mediated deregulated TCF/β-catenin signaling, upregulated expression of the p14^{ARF} tumor suppressor, and decreased expression of MDM2. Activation of this GSK3β-p53 axis stimulated a survivin-dependent apoptotic response mediated by p53, with inhibition of early tumor growth, in vivo.

### Example 1

### Purvalanol A is a GSK3β Antagonist

To determine if Purvalanol A had an inhibitory effect on kinases other than p34^{cdc2} the following experiment was performed using p53^{+/+} HCT116 colorectal carcinoma cells were maintained in culture according to published protocols (see Beltrami et al., J Biol. Chem., 279:2077-2084 (2004)). Cells were incubated with GSK3β inhibitors lithium chloride (LiCl) (30 mM, Sigma, St. Louis, Mo.), 4-Benzyl-2-methyl-1,2,4-thiadiazolidine-3,5-dione (thiadiazolidinone-8 or TDZD) (20 μM, Calbiochem, San Diego Calif.,), or Purvalanol A (10 μM, Calbiochem), harvested after 6 or 12 hours, and immunoprecipitated with an antibody to GSK3β, as described above. Immune complexes were mixed with a synthetic peptide containing a GSK3β phosphorylation site derived from the sequence of glycogen synthase (Upstate Biotechnology) in the presence of 10 μCi of γ-³²P-ATP (Amersham Corp., Piscataway, N.J.), after 30 min incubation at 30° C., the reaction mixtures were centrifuged briefly, and 15 μl of the supernatant was spotted onto P81 phosphocellulose paper (Whatman Inc., Clifton, N.J.). The membrane was washed three times with 175 mM phosphoric acid, rinsed once in acetone, and dried. Changes in peptide phosphorylation under the various conditions tested were determined by scintillation counting. Data were analyzed using the unpaired t test on a Graphpad Prism software package for Windows (Graphpad Inc., San Diego, Calif.). A P value of 0.05 was considered as statistically significant.

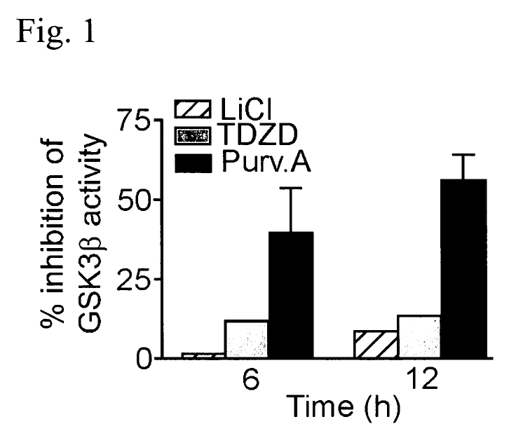

FIG. 1 shows the results of immunoprecipitation and GSK3β kinase assay using glycogen synthase-derived peptide substrate. Known p34^{cdc2} antagonist Purvalanol A potently inhibited GSK3β kinase activity in HCT116 cells after 6 and 12 hours of treatment. Established GSK3β inhibitors LiCl or TDZD also suppressed GSK3β activity in HCT116 cells. Purvalanol A was nearly five-fold more potent inhibitor of GSK3β than LiCl or TDZD at the concentrations used.

These surprising results indicate that Purvalanol A is a more potent inhibitor of GSK3β activity in p53^{+/+} colorectal cancer cells than certain established GSK3β inhibitors.

### Example 2

### Purvalanol A Induces Cell Cycle-Independent Induction of p53

To determine the effects of Purvalanol A on colorectal cancer cells and if those effects are dependent on p53, experiments were performed using p53^{+/+} or p53^{-/-} HCT116 colorectal carcinoma cells that were kindly provided by Dr. B. Vogelstein (Johns Hopkins University, Baltimore, Md.) and maintained in culture according to published protocols (see Beltrami et al., J Biol. Chem., 279:2077-2084 (2004)). Cells were incubated with increasing concentrations (0-20 μM) of the p34^{cdc2} antagonists Purvalanol A, olomoucine, or alsterpaullone (all from Calbiochem) for 6-48 hours at 37° C. Cells were collected and centrifuged at 3,000 rpm for 5 minutes at 4° C., and washed in PBS (pH 7.2). The cell pellet was solubilized in lysis buffer (20 mM Tris, pH 7.2, 0.5% deoxycholate, 1% Triton X-100, 0.1% SDS, 150 mM NaCl, 1 mM EDTA) containing proteases inhibitors (Complete, Roche Applied Science) for 30 min at 4° C. The lysate was cleared by centrifugation for 15 minutes, and protein concentration was evaluated using a Bio-Rad protein assay system (Bio-Rad Laboratories, Hercules, Calif.). Samples (30 μg) were separated by SDS-polyacrylamide gel, transferred to nylon membranes (Millipore), and incubated with primary antibodies to p53 (Oncogene Research Products, FL-393, Santa Cruz Biotechnology, Inc., Santa Cruz, Calif.), p21^{Waf1/Cip1} (Oncogene Research Products), XIAP (BD Transduction Laboratories), survivin (NOVUS Biologicals, Littleton, Colo.), MDM2 (Santa Cruz Biotechnology, Inc.), p14^{ARF} (NOVUS Biologicals), caspase-3 (Cell Signaling Technology), cleaved caspase-3 (Cell Signaling Technology), β-catenin (BD Transduction Laboratories), cyclin B1 (GNS1, Santa Cruz Biotechnology, Inc.), GSK3β (Santa Cruz Biotechnology, Inc.), or β-sactin (Sigma) with detection of reactive bands by chemiluminescence (Amersham). Cells were analyzed for hypodiploid DNA content by propidium iodide staining and flow cytometry as described in O'Connor et al., Cancer Cell, 2:43-54 (2002). Chemicals whose source is not explicitly listed were or can be obtained from Sigma.

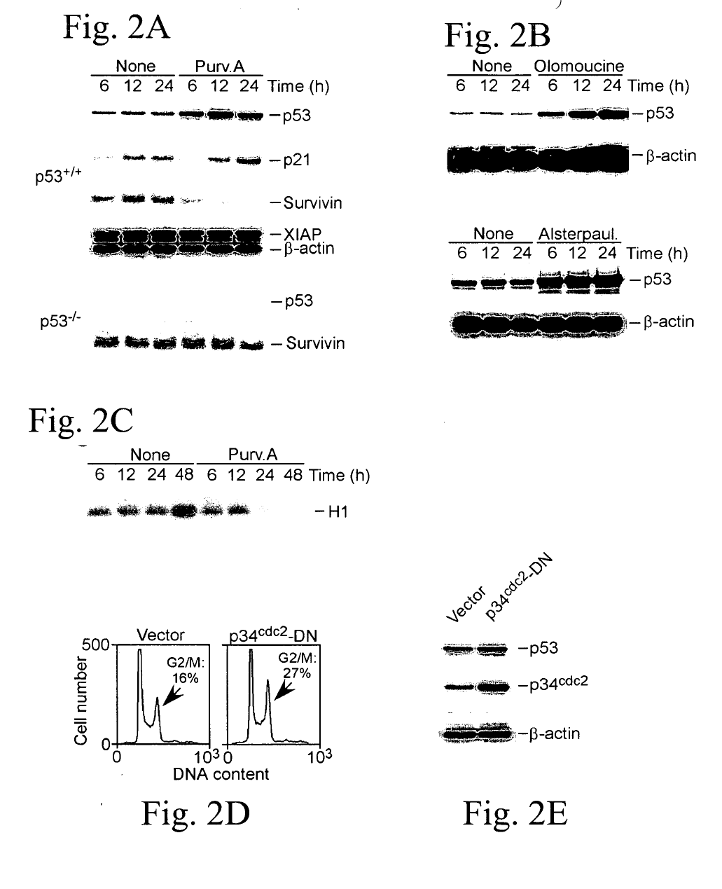

FIG. 2A, upper panel, shows that treatment of p53^{+/+} HCT116 colorectal cancer cells with Purvalanol A resulted in time-dependent accumulation of p53 that was detectable 6 hours after stimulation and remained elevated for 24 hours (Purv. A), as compared with vehicle-treated cultures (None). Increased p53 expression coincided with upregulation of p21^{Waf1/Cip1} and loss of survivin expression, two genes that are positively or negatively regulated by p53, respectively; however, no changes in the expression of another IAP family protein, XIAP, or β-actin were observed with or without Purvalanol A. See FIG. 2A, upper panel. FIG. 2A, lower panel, shows that Purvalanol A did not modulate the expression of p53-reactive material in control p53^{-/-} HCT116 cells, and no significant changes in survivin levels were observed in these cells.

FIG. 2B shows that treatment of p53^{+/+} HCT116 cells with two other structurally unrelated p34^{cdc2} antagonists, olomoucine and alsterpaullone, also resulted in elevation of p53 levels with the same kinetics as observed with Purvalanol A.

Additional experiments showed that Purvalanol A induction of p53 was independent of mitotic deregulation. As shown in FIG. 2C (lanes labeled None), p34^{cdc2} that was immunoprecipitated from HCT116 cells treated with vehicle for 6 to 48 hours consistently phosphorylated histone H1. FIG. 2C also shows (lanes labeled Purv. A), that after 6 and 12 hours of treatment with Purvalanol A, HCT116 cells show no apparent inhibition of p34^{cdc2} kinase activity. These earlier time points coincide with the peak upregulation of p53 at 6 and 12 hours shown in FIG. 2A. In contrast, 24 and 48 hours of treatment with Purvalanol A did appear to suppress histone H1 phosphorylation, as shown in FIG. 2C.

In another experiment, HCT116 cells were transfected with a construct expressing a p34^{cdc2} Asp¹⁴⁶→Asn dominant negative mutant (p34^{cdc2} DN mutant) that is known to interfere with mitotic progression (O'Connor et al., Proc. Natl. Acad. Sci. USA., 97:13103-13107 (2000)). As shown in FIG. 2D, DNA content analysis and flow cytometry indicated that expression of the p34^{cdc2} DN mutant resulted in cell cycle arrest at G2/M. Expression of p34^{cdc2} DN was also associated with accumulation of p34^{cdc2}-reactive material as shown by Western blotting in FIG. 2E. However, FIG. 2E also shows that no changes in p53 expression were observed in HCT116 cells transfected with control vector or p34^{cdc2} DN mutant.

These results indicate that treatment with Purvalanol A, olomoucine, and alsterpaullone induce p53 expression in colorectal cancer cells and that p53 induction is independent of cell-cycle (mitotic) deregulation.

### Example 3

### GSK3β Modulates p53 In Vivo

To explore the link between GSK3β inhibition and p53 modulation, in vivo, HCT116 cells were treated with Li, TDZD or SB216763, and analyzed cells by Western blotting. p53^{+/+} or p53^{-/-} HCT116 cells were incubated with the following GSK3β inhibitors, LiCl (Sigma, 30 mM), thiadiazolidinone-8 (TDZD) (Calbiochem, 0-20 μM), or SB216763 (Sigma, 20 μM), cells were harvested after 6-24 hours, and samples were analyzed by Western blotting as described in Example 2.

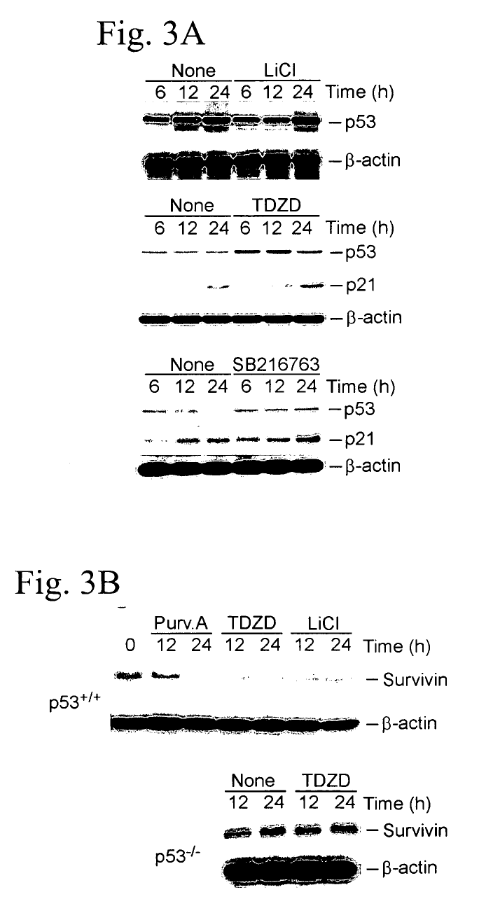

FIG. 3A shows that TDZD and SB216763 caused a strong induction of p53 in p53^{+/+} HCT116 cells, which was detectable after 6 hours and sustained after 24 hours of incubation with GSK3β inhibitor. This induction was associated with p53 transcriptional activity (not shown) and prominent upregulation of p21^{Waf1/Cip1}, that is also shown in the Western blots of FIG. 3A. Consistent with the kinase assay data described in Example 2, LiCl was considerably less effective and caused elevation of p53 levels only at 24 hours after treatment (FIG. 3A).

The ability of GSK3β inhibitors to control p53-mediated downstream effectors of apoptosis was shown in p53^{+/+} and p53^{-/-} HCT116 cells. As shown by FIG. 3B, treatment with Purvalanol A, TDZD or LiCl all resulted in time-dependent downregulation of survivin in p53^{+/+} HCT116 cells. Purvalanol A and TDZD produced the strongest responses. In contrast, no changes in survivin expression were observed in p53^{-/-} HCT116 cells treated with vehicle or TDZD at the same time intervals tested. See FIG. 3C.

These results indicate that GSK3β inhibitors elevate p53 expression and activate a p53 pathway that upregulates p21^{Waf1/Cip1} and downregulates survivin.

### Example 4

### Molecular Targeting of GSK3β Modulates p53 Expression

To confirm the link between GSK3β inhibition and p53 modulation in vivo, p53^{+/+} HCT116 cells were transfected with wild-type GSK3β or a kinase-dead GSK3β dominant negative mutant (GSK3β DN mutant), previously described in Pap and Cooper, J. Biol. Chem., 273:19929-19932 (1998). Non-transformed wild-type (WT) or GSK3β^{-/-} primary murine embryonic fibroblasts (MEF) cells, described in Hoeflich et al., Nature, 406:86-90 (2000), were maintained in culture in DMEM supplemented with 10% FCS, 100 U/ml penicillin, and 100 μg/ml streptomycin (all from Invitrogen) in 5% CO₂ at 37° C. Transfected HCT116 cells or non-transformed MEF cells were treated with Purvalanol A (10 mM). Western blotting was performed as described in Example 2, to evaluate the expression of p53, p21^{Waf1/Cip1} or β-actin at 6-24 hour intervals.

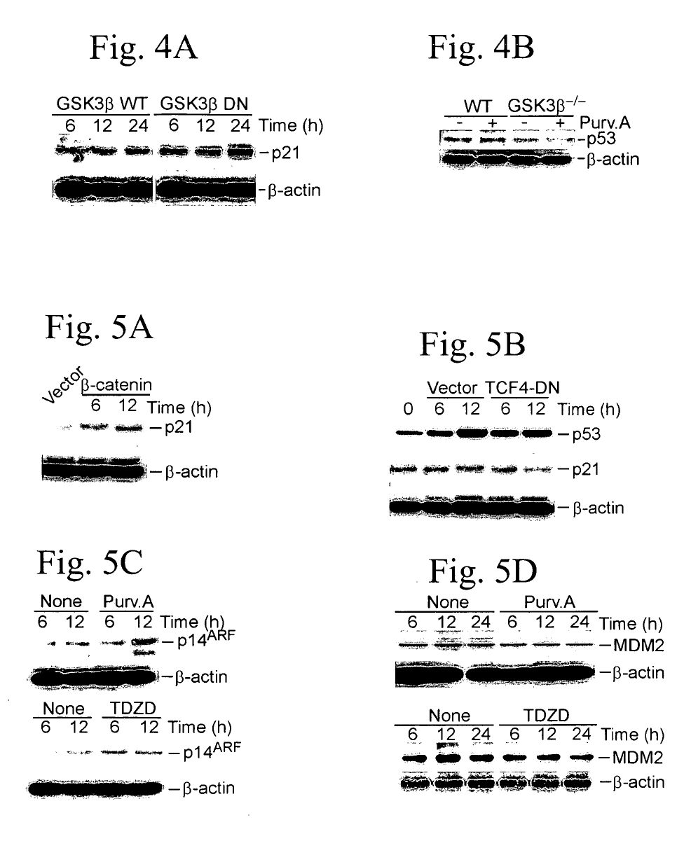

Transfection of HCT116 cells with wild-type GSK3β did not affect p53 levels (not shown) and did not effect p21^{Waf1/Cip1} expression. See FIG. 4A. In contrast, FIG. 4A also shows that expression of GSK3β DN mutant resulted in sustained upregulation of p21^{Waf1/CiP1} throughout the 24-hour time interval in HCT116 cells. FIG. 4B shows that non-immortalized primary GSK3β^{-/-} MEF cells exhibit constitutively elevated levels of p53, as compared with wild-type MEF. Furthermore, the addition of Purvalanol A did not further modulate p53 expression in wild-type or GSK3β^{-/-} MEF cells, as shown by FIG. 4B.

These results indicate that genetic inactivation of GSK3β elevates p53 levels.

### Example 5

### Mechanisms of p53 Induction by GSK3β Inhibition

GSK3β has been shown to regulate β-catenin activity (Polakis, P. Genes Dev., 14:1837-1851, (2000)). Therefore, experiments were performed to determine if forced expression of β-catenin would reproduce the effect of GSK3β inhibitors on p53 function. Source and maintenance of p53^{+/+} HCT116 colorectal carcinoma were as described in Example 1. HCT116 cells were transfected with (i) wild-type β-catenin or (ii) cDNA of a truncated dominant negative mutant of TCF-4 (TCF-4 DN mutant), which is missing the N-terminal 31 amino acids of wild-type TCF-4. Kim et al., Lancet, 362:205-209 (2003). Transfected cells were treated with Purvalanol A (10 μM), and analyzed for changes in expression of p53, p21^{Waf1/Cip1} or β-actin at 6-12 hour time intervals, by Western blotting.

FIG. 5A shows that transfection of wild-type β-catenin in HCT116 cells resulted in time-dependent upregulation of p21^{Waf1/Cip1}, consistent with induction of p53-dependent transcription. Conversely, as shown in FIG. 5B, expression of a TCF-4 DN mutant that lacks the DNA binding domain and interferes with TCF/β-catenin signaling blunted Purvalanol A induction of p53 at 12 hour, and considerably reduced p21^{Waf1/Cip1} expression in these cells, relative to empty vector control cells.

GSK3β inhibitor-mediated induction of p53 also resulted in the upregulation of the tumor suppressor and p53 regulator, p14^{ARF}. FIG. 5C shows that exposure of HCT116 cells to Purvalanol A or TDZD resulted in upregulation of p14^{ARF}, relative to vehicle treated control cells. Purvalanol A and TDZD treatments reduced expression of the negative p53 regulator, murine double minute 2 gene (MDM2), in HCT116 cells as shown in FIG. 5D.

These results indicate that GSK3β inhibitors elevate p53 in colorectal cancer cells by a mechanism that involves (a) deregulation of TCF/β-catenin signaling (b) upregulation of the p14^{ARF} tumor suppressor, and (c) decreased expression MDM2 level.

### Example 6

### Inhibition of GSK3β Induces p53-Dependent Apoptosis

The functional consequences of GSK3β inhibition were explored. Source and maintenance of p53^{+/+} and p53^{-/-} HCT116 colorectal carcinoma cells were as described in Example 2. HCT116 cells were treated with Purvalanol A (0-20 μM), harvested after 48 hours and analyzed for hypodiploid DNA content by propidium iodide staining and flow cytometry, as described in O'Connor et al., Cancer Cell, 2:43-54 (2002). Alternatively, cells were incubated with GSK3β inhibitors LiCl (30 mM) or TDZD (20 μM), harvested after 48 hours and analyzed for cell viability (propidium iodide, red channel) and active caspase-3/7 activity (green channel, CASPATAG®, from Chemicon International Inc., Temecula, Calif.), by multiparametric flow cytometry as reported previously in Kim et al., Lancet, 362:205-209 (2003). In some experiments, Purvalanol A (10 μM)-treated p53^{+/+} HCT116 cells were harvested after a 6-48 hour culture at 37° C., and extracts were separated by SDS gel electrophoresis and analyzed with an antibody to caspase-3, by Western blotting.

To determine the role of survivin in modulation of Purvalanol A-induced apoptosis, p53^{+/+} HCT116 cells were transduced with replication-deficient adenoviruses encoding GFP (pAd-GFP) or survivin (pAd-Survivin) at multiplicity of infection (moi) of 50 for 24 hour. See Mesri et al., J. Clin. Invest., 108:981-990 (2001). Transduced cultures were treated with vehicle or Purvalanol A (10 μM), and analyzed for hypodiploid DNA content by propidium iodide staining and flow cytometry after an additional 24 hour incubation at 37° C. In other experiments, acute ablation of survivin in p53^{-/-} HCT116 was carried out by RNA interference (RNAi). Cells were transfected with control (VIII) or survivin-derived (S4) double stranded (ds) RNA oligonucleotides (50 nM) using oligofectamine (3 μl/well) reagent in 1 ml of optiMEM medium (both from Invitrogen). Survivin-derived (ds)RNA oligonucleotides and RNAi protocols are described in Beltrami et al., J Biol. Chem., 279:2077-2084 (2004).

Four hours after transfection, cells were replenished with complete growth medium containing 125 μl of McCoy's medium containing 30% FCS, 300 U/ml penicillin, and 300 μg/ml streptomycin. For double transfection experiments, p53^{+/+} HCT116 cells were loaded twice with dsRNA oligonucleotides at 24-hour intervals between each transfection, and incubated with vehicle or a suboptimal concentration of Purvalanol A (5 μM) for an additional 24 hour culture at 37° C. before analysis of hypodiploid DNA content by flow cytometry, and survivin expression by Western blotting. Western blotting was performed as described in Example 2.

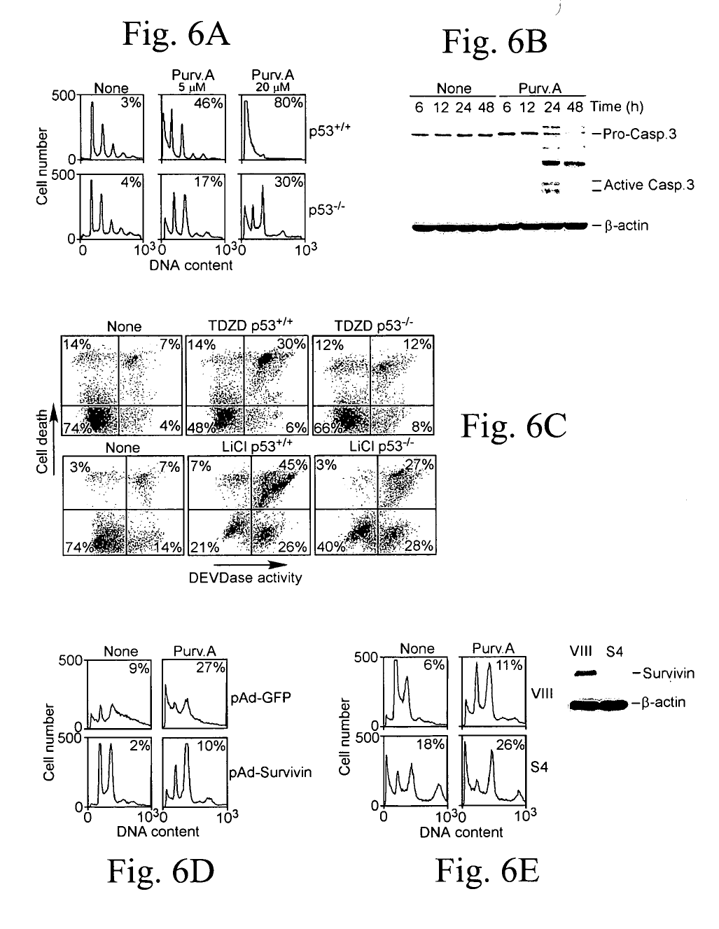

FIG. 6A, upper panels, shows that treatment of p53^{+/+} HCT116 cells with Purvalanol A resulted in concentration-dependent induction of apoptosis as indicated by hypodiploid DNA content and flow cytometry. In contrast, the lower panels of FIG. 6A show that p53^{-/-} HCT116 cells were largely resistant to Purvalanol A-induced cell death, and exhibited a sustained G2/M arrest under these conditions. The Western blot shown in FIG. 6B indicates that Purvalanol A treatment of p53^{+/+} HCT116 cells caused time-dependent cleavage of 32 kD proform caspase-3 to active caspase-3 fragments of 17 and 19 kD, as compared with vehicle-treated cultures.

To clarify the role of GSK3β inhibition in p53-dependent apoptosis, HCT116 cells were treated with TDZD or LiCl and monitored changes in cell death. Results are shown in FIG. 6C. In these experiments, LiCl or TDZD reproduced caspase-dependent apoptosis in p53^{+/+} HCT116 cells, by multiparametric flow cytometry of DEVDase activity and plasma membrane integrity. See the two center column panels of FIG. 6C. In contrast, LiCl or TDZD were considerably less effective at inducing apoptosis in p53^{-/-} HCT116 cells. See right hand column panels of FIG. 6C. Quantification of the cell death response in these experiments revealed that TDZD increased the fraction of apoptotic cells by ˜4.5 fold in p53^{+/+} cultures and only by 1.7 fold in p53^{-/-} cells.

Downregulation of survivin in response to p53 activation (shown in FIG. 2A) contributed to apoptosis following GSK3β inhibition. FIG. 6D shows that transduction of p53^{+/+} HCT116 cells with a replication-deficient adenovirus encoding survivin (pAd-Survivin) completely reversed Purvalanol A-induced apoptosis to background levels of untreated cultures; whereas expression of pAd-GFP did not reverse Purvalanol A-induced apoptosis in p53^{+/+} HCT116 cells, by hypodiploid DNA content and flow cytometry. In reciprocal experiments, survivin was acutely ablated in p53^{-/-} HCT116 cells by RNA interference (RNAi), and monitored their sensitivity to Purvalanol A-induced apoptosis. The Western blots (right hand panels) shown in FIG. 6E indicate that transfection of HCT116 cells with a survivin-specific dsRNA oligonucleotide (S4) resulted in nearly complete ablation of survivin levels, as compared with cultures transfected with an unrelated dsRNA oligonucleotide (VIII). This acute ablation of survivin by S4 transfection restored sensitivity of p53^{-/-} HCT116 cells to Purvalanol A-induced apoptosis, and enhanced cell death by 2.5-fold as compared with VIII transfectants treated with Purvalanol A, as shown in the left hand four panels of FIG. 5E.

These results indicate that inhibition of GSK3β stimulated a survivin-dependent, p53-mediated apoptotic response in colorectal cancer cells.

### Example 7

### Purvalanol A Demonstrates Anti-Colorectal Cancer Activity In Vivo

The in vivo anti-tumor efficacy of Purvalanol A was tested using a xenograft tumor model with in vivo adapted p53^{+/+} HCT116 cells. In vivo adapted p53^{+/+} HCT116 cells (2.5×10⁶) were injected in the flank of CB17 SCID/beige mice (Taconics, Germantown, N.Y.) and allowed to form palpable solid tumors for 4-6 days. When tumors reached ˜50 mm in volume, animals were randomized in 3 groups (4 animals/group; two tumors/animal) and administered no treatment, vehicle, or Purvalanol A (20 mg/kg) daily for 5 consecutive days as intraperitoneal (i.p.) injections (200 μl/injection). Tumor measurements were taken daily for a week with a caliper. Tumor volume was calculated according to the formula ½ [length (mm)]×[width (mm)]². No signs of systemic toxicity or weight loss were observed throughout the Purvalanol A treatment regimen.

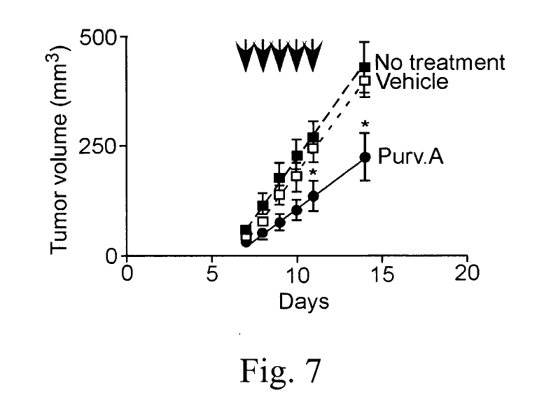

FIG. 7 shows the results of this experiment. Adapted HCT116 cells in CB-17 SCID/beige mice gave rise to rapidly growing solid tumors over a one-week period. The effect of Purvalanol A was evaluated during the first week after establishment of palpable masses (˜50 mm³ volume). Administration of vehicle (open squares) to tumor-bearing animals did not significantly affect exponential growth of adapted HCT116 cells in vivo, as compared with untreated mice (filled-in squares). See FIG. 7. In contrast, daily administration of Purvalanol A (25 mg/kg i.p.) for 5 days significantly inhibited early tumor growth. See filled-in circles in FIG. 7. A higher concentration of Purvalanol A at a comparable regimen (50 mg/kg i.p. injection daily for 5 days) did not result in further inhibition of tumor growth within the same time interval. At these Purvalanol A concentrations, animals exhibited no signs of local or systemic toxicity or weight loss throughout the treatment.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating an individual with colorectal cancer, the method comprising:
determining whether colorectal cancer cells of the individual express functional p53 protein; and,
if the cells express functional p53 protein, administering to the individual an effective amount of a glycogen synthase kinase 3β (GSK3β) inhibitor.

2. The method of claim 1, wherein determining whether the colorectal cancer cells express functional p53 protein comprises reviewing the individual's medical history.

3. The method of claim 1, wherein determining whether the colorectal cancer cells express functional p53 protein comprises:
obtaining from the individual a sample comprising colorectal cancer cells; and
assaying expression of functional p53 protein in the cells in the sample.

4. The method of claim 1, wherein determining whether the colorectal cancer cells express functional p53 protein comprises detecting the presence of mutant or wild-type forms of a p53 gene or protein.

5. The method of claim 1, wherein determining whether the colorectal cancer cells express functional p53 protein comprises assaying one or more functions of p53 protein in the cells.

6. The method of claim 5, wherein assaying one or more functions of p53 protein comprises assaying p53 transcription factor activity in the cells.

7. The method of claim 1, wherein the GSK3β-inhibitor is Purvalanol A.

8. The method of claim 1, wherein the GSK3β-inhibitor is selected from the group consisting of olomoucine; lithium chloride (LiCl); alsterpaullone; and kenpaullone.

9. The method of claim 1, wherein the GSK3β-inhibitor is an indirubin.

10. The method of claim 1, wherein the GSK3β-inhibitor is selected from the group consisting of indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin; and 5-bromoindirubin.

11. The method of claim 1, wherein the GSK3β-inhibitor is selected from the group consisting of benzyl-2-methyl-1,2,4-thiadiazolidine-3,5-dione (TDZD-8); 2-thio(3-iodobenzyl)-5-(1-pyridyl)-[1,3,4]-oxadiazole (GSK3 inhibitor II); 2,4-dibenzyl-5-oxothiadiazolidine-3-thione (OTDZT); (2′Z,3′E)-6-Bromoindirubin-3′-oxime (BIO); α-4-Dibromoacetophenone, 2-Chloro-1-(4,5-dibromo-thiophen-2-yl)-ethanone, N-(4-Methoxybenzyl)-N′-(5-nitro-1,3-thiazol-2-yl)urea (AR-A014418), H-KEAPPAPPQSpP-NH2 (L803; SEQ ID NO:1) and Myr-N-GKEAPPAPPQSpP-NH2 (L803-mts; SEQ ID NO:2).

12. A method of identifying a candidate for colorectal cancer treatment with a glycogen synthase kinase 3β (GSK3β) inhibitor, the method comprising:
identifying an individual having colorectal cancer cells;
obtaining one or more colorectal cancer cells from the individual; and
assaying expression of functional p53 protein in the colorectal cancer cells, wherein:
(i) if the screening indicates that the individual's colorectal cancer cells express functional p53 protein, the method further comprises classifying the individual as a candidate for treatment with a GSK3β inhibitor; and
(ii) if the screening indicates that the individual's colorectal cancer cells do not express functional p53 protein, the method further comprises classifying the individual as someone who is not a candidate for colorectal cancer treatment with a GSK3β inhibitor.

13. The method of claim 12, wherein assaying expression of functional p53 protein in the colorectal cancer cells comprises detecting the presence of mutant or wild-type forms of a p53 gene or protein.

14. The method of claim 12, wherein determining whether the colorectal cancer cells express functional p53 protein comprises assaying one or more functions of p53 protein in the cells.

15. The method of claim 12, wherein assaying one or more functions of p53 protein in the cells comprises assaying p53 transcription factor activity in the cells.
